(19) United States
(12) Patent Application Publication
(10) Pub. No.: US 2012/0041256 A1
(43) Pub. Date: Feb. 16, 2012

(54) APPARATUS FOR PROVIDING SHORT-TERM CARDIAC SUPPORT

(75) Inventors: Stephen Large, Cambridge (GB); Samer Nashef, Cambridge (GB)

(73) Assignee: PAPWORTH HOSPITAL NHS FOUNDATION TRUST, Cambridge, Cambridgeshire (GB)

(21) Appl. No.: 13/145,809

(22) PCT Filed: Jan. 21, 2010
(86) PCT No.: PCT/GB2010/050081

§ 371 Date: Oct. 3, 2011

(30) Foreign Application Priority Data
Jan. 22, 2009 (GB) 0901038.0

(51) Int. Cl. A61M 1/10

(52) U.S. Cl. 600/17; 600/16

(57) ABSTRACT

An apparatus provides short-term cardiac support after cardiac surgery using a cardiopulmonary bypass (CPB) pump (or heart-lung machine) having a pump coupled to a pump controller for pumping oxygenated blood through a cannula into the patient The pump controller controls the pump in a first mode to generate pulsatile flow of the oxygenated blood to the patient, and in a second mode to provide counter-pulsatile flow of blood through the cannula synchronized with the heart of the patient, whereby, when the heart of the patient is in diastole, the pump forces blood from the cannula into the patient, and when the heart is in systole, the pump removes blood from the patient via the cannula.

### FIELD OF THE INVENTION

This invention relates to apparatus for providing short-term cardiac support, particularly, though not exclusively, for providing short term cardiac support towards the end of a heart operation, after surgery on the heart has been completed, but before wound closure.

### BACKGROUND OF THE INVENTION

Cardiopulmonary bypass (CPB) is a technique that temporarily takes over the function of the heart and lungs during surgery, maintaining the circulation of blood and the oxygen content of the body. A CPB pump itself is often referred to as a Heart-Lung Machine or the Pump. Cardiopulmonary bypass pumps are operated by allied health professionals known as perfusionists in association with surgeons who connect the pump to the patient's body.

CPB mechanically circulates and oxygenates blood for the body while bypassing the heart and lungs. It uses a heart-lung machine to maintain perfusion to other body organs and tissues while the surgeon works in a bloodless surgical field. The surgeon places a cannula (the venous cannula) in the right atrium, venae cavae, or femoral vein to withdraw blood from the body. The venous cannula is connected to tubing filled with isotonic crystalloid solution. Blood that is removed from the body by the venous cannula is filtered, oxygenated, cooled or warmed, and then returned to the body via a return cannula (the arterial cannula). The arterial cannula used to return oxygenated blood is usually inserted in the ascending aorta, but it may be inserted in another artery. During the final stages of surgery the surgeon will allow the heart to start beating again and take over the circulation from the heart-lung machine. The patient is then disconnected from the heart-lung machine first by removing the venous cannula that withdraws blood from the patient, and then the arterial cannula usually located in the aorta.

A number of different types of heart-lung machines are known. In general, however, they include a pump, which may be a roller (or peristaltic) pump or a centrifugal pump and an oxygenator, which removes carbon dioxide from the venous blood and adds oxygen. They also usually include a reservoir for storing the blood and a mechanism for anticoagulants to be provided to stop the blood clotting. The pump may function continuously or in a pulsatile fashion, mimicking the heartbeat. Different mechanisms for achieving optimal pulsatile blood flow in such a machine are known. For example, in PCT Application No. WO 97/40865 there is described a mechanism of using a bladder which can be compressed and released to simulate the human heart's action to provide the pulsatile blood flow, compression of the bladder being controlled to simulate the human heartbeat. U.S. Pat. No. 5,928,179 describes a mechanism whereby an automatic proportioning valve is used to cyclically switch blood flow between the arterial supply line and a recycle line, which returns the blood to the reservoir of the machine. This patent also suggests that the patient's own heartbeat can be used to control the pulsing cycle, that heartbeat either being pre-recorded from the patient prior to the surgery commencing, or, once the heart has been re-started, using the actual heartbeat to synchronize the machine so that the pulsing is in phase with the heart, thereby allowing the amount of blood provided by the machine to be gradually reduced to aid the in weaning the patient off the heart-lung machine.

It will be apparent that at the conclusion of the surgery, the heart must be working properly to take over the task of maintaining the circulation and to allow the operation is to be completed. If it is not, then the patient may require cardiac support using an intra-aortic balloon. An intra-aortic balloon is usually inserted into the femoral artery near the groin and advanced towards the arch of the aorta. The balloon is then attached to a pump console (intra-aortic balloon pump or IABP) which inflates it, using helium, in diastole (when the heart is relaxing) and deflates it in systole (when the heart is contracting). By deflating in systole, it reduces the blood pressure in the aorta and thus reduces the afterload against which the heart has to pump, making the heart pumping action more effective. By inflating is diastole, it increases the pressure in the aorta and thus improves coronary perfusion, providing more oxygenated blood to the heart muscle itself.

The intra-aortic balloon pump is connected via a computer-controlled mechanism to the patient's electrocardiogram (ECG) and to a pressure transducer which measures the pressure at the end of the intra-aortic balloon in the aorta. Using one or the other of these measurements, the intra-aortic balloon pump synchronizes the inflation and deflation of the intra-aortic balloon with the heartbeat to obtain the desired effects. Helium is used to inflate the balloon because its low viscosity allows it to travel quickly through the long connecting tubes, and has a lower risk of causing a harmful embolism should the balloon rupture while in use.

However, since the balloon is placed into the femoral artery and aorta it can provoke ischaemia. At highest risk is the leg which is supplied by the femoral artery, but other arteries that originate from the aorta and the aorta itself may be at risk from balloon insertion and manipulation. Other possible complications are cerebral or systemic embolism during insertion, infection, dissection of the aorta or iliac artery, perforation of the artery and hemorrhage in the mediastinum, thorax or abdomen. Mechanical failure of the balloon itself is also a risk which may require vascular surgery to remove it in such circumstances.

It can thus be seen that the decision to use an intra-aortic balloon is not one that can be taken lightly, since it requires the patient then to be connected to the pump and gas supply, and the balloon may require further surgery to remove. As a consequence, once inserted, the balloon may remain in situ for several days and the patient will be bed-bound during that time. Accordingly, the surgeon, when deciding whether to insert an intra-aortic balloon needs to balance the immediate benefits of supporting the heart in the short term after surgery with the disadvantages of using the intra-aortic balloon as outlined above.

### BRIEF SUMMARY OF THE INVENTION

The present invention therefore seeks to provide an apparatus for providing short-term cardiac support at the end of a heart operation in which a heart-lung machine is used.

Accordingly, in a first aspect, the invention provides an apparatus for providing short-term cardiac support after cardiac surgery using a cardiopulmonary bypass (CPB) pump (or heart-lung machine), the apparatus comprising a heart-lung machine having a pump coupled to a pump controller for pumping oxygenated blood through a first cannula into the patient, wherein the pump controller controls the pump in a first mode to generate pulsatile flow of the oxygenated blood to the patient, characterized in that the pump controller is further operable to control the pump in a second mode to provide counter-pulsatile flow of blood through the first cannula synchronized with the heart of the patient, whereby, when the heart of the patient is in diastole, the pump forces blood from the first cannula into the patient, and when the heart is in systole, the pump removes blood from the patient via the first cannula.

In one embodiment, the heart-lung machine further comprises at least one second cannula for positioning in a patient for removing venous blood therefrom, the second cannula being connected to a reservoir, and an oxygenator coupled to the reservoir for receiving venous blood therefrom and for removing carbon dioxide from the venous blood and transferring oxygen into the blood to produce the oxygenated blood.

In a second aspect, the invention provides an apparatus for causing a heart-lung machine to provide short term cardiac support after cardiac surgery using a cardiopulmonary bypass (CPB) pump (or heart-lung machine), the heart-lung machine having a pump coupled to a pump controller for pumping oxygenated blood through a first cannula into the patient, wherein the pump controller controls the pump in a first mode to generate pulsatile flow of the oxygenated blood to the patient, characterized in that the apparatus comprises means for causing the pump controller to control the pump in a second mode to provide counter-pulsatile flow of blood through the first cannula synchronized with the heart of the patient, whereby, when the heart of the patient is in diastole, the pump forces blood from the first cannula into the patient, and when the heart is in systole, the pump removes blood from the patient via the first cannula.

Preferably, the pump controller includes at least a first input for receiving information relating to the actual heartbeat of the patient and a second input for receiving control signals from an operator interface, the pump controller being operable to use the information relating to the actual heartbeat of the patient and the control signals to control the pump in the second mode to synchronize the counter-pulsatile flow of the blood to the heart of the patient.

The pump controller may receive information relating to the actual heartbeat of the patient from an electrocardiogram or from a pressure sensor sensing the blood pressure in the aorta, and may further receive information relating to the operation of a pacemaker fitted to the patient.

When in the second mode, the pump is preferably arranged to move between 0 and 200 ml (more preferably 0-100 ml) of blood through the first cannula when counter-pulsating, the amount of blood to be moved through the first cannula preferably being controlled via the control signals from the operator interface.

In one embodiment, the pump is a roller-type pump.

In a third aspect, the invention provides the use of an apparatus as described above, for providing short term cardiac support after a heart has been restarted following surgery.

According to a fourth aspect, the invention provides a method of using a cardiopulmonary bypass (CPB) pump (or heart-lung machine), the method comprising:
controlling a pump in a first mode to generate pulsatile flow of oxygenated blood through a cannula;
receiving signals from at least one of:
   an electrocardiogram;
   a pressure transducer; and
   a pacemaker;
controlling the pump in a second mode to generate counter-pulsatile flow of blood through the cannula synchronized with the signals from the electrocardiogram, the pressure transducer and/or the pacemaker.

When in the second mode, the pump is preferably arranged to move between 0 and 200 ml of blood through the cannula when counter-pulsating, the amount of blood to be moved through the cannula preferably being controlled via the control signals from the operator interface.

### BRIEF DESCRIPTION OF THE DRAWINGS

One embodiment of the invention will now be more fully described, by way of example, with reference to the drawings, of which:

FIG. 1 shows a schematic diagram of an apparatus according to one embodiment of the present invention connected to the heart of a patient.

### DETAILED DESCRIPTION OF THE DRAWINGS

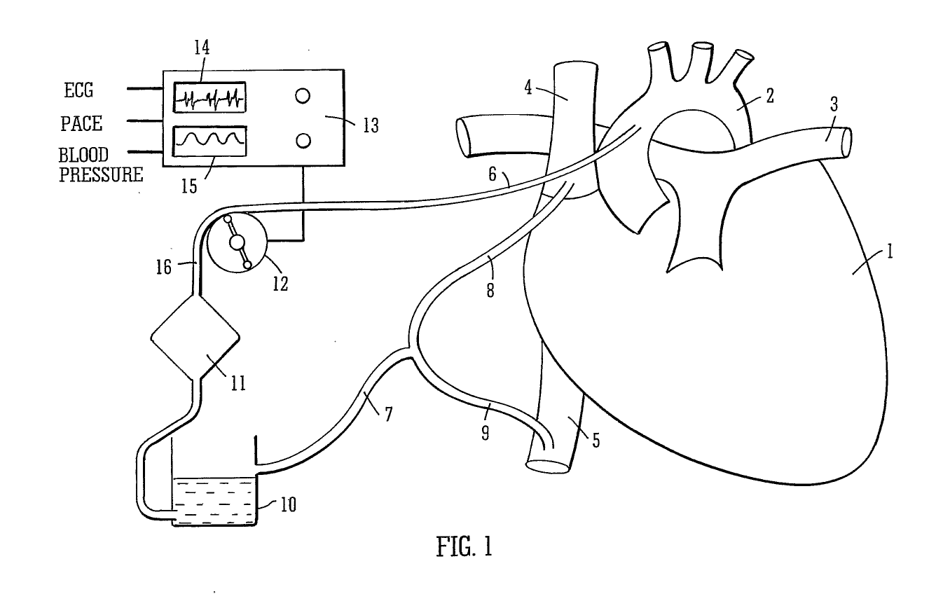

Thus, as shown in FIG. 1, a heart 1 of a patient includes the aorta 2, the pulmonary artery 3 and the superior vena cava 4 and inferior vena cava 5 (the remaining parts of the heart are not shown for simplicity). The venae cavae 4, 5 return deoxygenated blood (venous blood) from the body back to the heart 1, the pulmonary artery 3 moves the venous blood from the heart 1 to the lungs to be oxygenated, and the aorta 2 moves the oxygenated blood (that has been returned to the heart 1 via the pulmonary vein) to the body.

When surgery is to be performed on the heart such that the heart must be stopped, a heart-lung machine is used to take the place of the heart and lungs of the patient. Thus, a first cannula 6 is inserted into the aorta 2 (usually into the ascending aorta, but sometimes into the femoral artery) and a second cannula 7, which may be split into two portions 8 and 9 is inserted into the superior vena cava 4 and inferior vena cava 5 as shown (although it could be inserted into the right atrium or femoral vein). In this way, the venous blood is removed by the second cannula 7 (via portions 8 and 9) and transferred to a reservoir 10 of the heart-lung machine. The oxygenated blood is then returned to the aorta 2 through the first cannula 6 thereby bypassing the pulmonary artery and vein and the lungs.

From the reservoir 10, the venous blood is passed to an oxygenator 11, where the carbon dioxide is removed and oxygen is transferred into the blood in known fashion. A pump 12, controlled by a pump controller 13, is used to generate pulsatile flow of the oxygenated blood through the first cannula 6 into the aorta 2. The pump 12 is preferably a roller pump in which a roller is caused to rotate in an orbital fashion to compress tubing 16 containing the oxygenated blood so as to cause it to move along the tubing 16 to the first cannula 6. Because of the cyclic motion of the roller pump 12 a pulsed movement of the blood into the aorta 2 is caused, simulating the beat of the heart. The pump controller 13 may include inputs to receive signals from an electrocardiogram providing the beat of the heart (when it is beating), from a pacemaker (if one is fitted), and/or from a blood pressure transducer sensing the blood pressure in the body. These signals may be displayed on the controller 13, as shown at display 14 (showing the electrocardiogram and the pacemaker signals superimposed on each other) and display 15 (showing the blood pressure). The pump controller 13 may also include user interface inputs (such as controls 17, 18) to enable an operator to control the pump to operate as desired. Other control inputs may, of course, also be provided.

The pump 12, whether rotary or not, is usually operable in reverse in case of an emergency, such as a blood clot or air bubble being detected in the first cannula 6. If such an alarm is raised, the pump is immediately controlled to drive in reverse so that the blood with the clot or air bubble is not emitted into the aorta, but is sucked back into the heart-lung machine. In such cases, there may well be a bypass provided in the machine so that the blood is directed straight to the reservoir 10.

Following the completion of the surgery on the heart, but before the end of the operation, as mentioned above, the heart will be re-started and the surgeon will consider whether it is working well enough to keep the patient alive. Sometimes, if the heart is not operating at maximum efficiency, it may be difficult to determine immediately whether the heart will recover fully within a short period of time, so that the operation can be completed, or if the heart will require the support of an intra-aortic balloon for a few days.

Using the apparatus of the invention, however, the heart may be put onto short term support before the operation is completed to help the heart recover fully and to thereby obviate the need to use an intra-aortic balloon in cases where only a short term support is required, rather than the several days that the intra-aortic balloon option usually provides. Thus, according to an embodiment of the invention, the pump controller 13 is switched into a second mode of operation in which the pump 12 is operated in reverse in synchronism with the heartbeat of the patient, so as to withdraw a small amount of blood back into the first cannula 6 from the aorta 2 while the heart is contracting (in systole) and to force a small amount of blood into the aorta 2 from the first cannula 6 when the heart is relaxing (in diastole). Thus, the heart-lung machine will be operating in the opposite phase to that of the heart, in the same manner as an intra-aortic balloon, to thereby provide support to the heart for a time before the end of the operation. Of course, if the surgeon determines that the heart will require longer term support, then an intra-aortic balloon can still be inserted.

Since this counter-pulsatile flow of blood only occurs through the first cannula 6 inserted into the aorta 2, the portions 8 and 9 of the second cannula 7 can be removed from the venae cavae 4 and 5 at this time. The amount of blood that is moved is usually between about 0 and 200 ml, the actual amount being controlled by the operator using the pump controller 13. Preferably, the amount is relatively small and is between about 0 and 50 ml and preferably between 0 and 15 ml. This control means that the amount of support that is provided can be decreased over time to help the heart recover to full efficiency.

It will be appreciated that although only one particular embodiment of the invention has been described in detail, various modifications and improvements can be made by a person skilled in the art without departing from the scope of the present invention.

1: An apparatus for providing short term cardiac support after cardiac surgery using a cardiopulmonary bypass (CPB) pump (or heart-lung machine), the apparatus comprising a heart-lung machine having a pump coupled to a pump controller for pumping oxygenated blood through a first cannula into the patient, wherein the pump controller controls the pump in a first mode to generate pulsatile flow of the oxygenated blood to the patient, wherein the pump controller is further operable to control the pump in a second mode to provide counter-pulsatile flow of blood through the first cannula synchronized with the heart of the patient, whereby, when the heart of the patient is in diastole, the pump forces blood from the first cannula into the patient, and when the heart is in systole, the pump removes blood from the patient via the first cannula.

2: An apparatus according to claim 1, wherein the heart-lung machine further comprises at least one second cannula for positioning in a patient for removing venous blood therefrom, the second cannula being connected to a reservoir, and an oxygenator coupled to the reservoir for receiving venous blood therefrom and for removing carbon dioxide from the venous blood and transferring oxygen into the blood to produce the oxygenated blood.

3: An apparatus for causing a heart-lung machine to provide short term cardiac support after cardiac surgery using a cardiopulmonary bypass (CPB) pump (or heart-lung machine), the heart-lung machine having a pump coupled to a pump controller for pumping oxygenated blood through a first cannula into the patient, wherein the pump controller controls the pump in a first mode to generate pulsatile flow of the oxygenated blood to the patient, wherein the apparatus comprises means for causing the pump controller to control the pump in a second mode to provide counter-pulsatile flow of blood through the first cannula synchronized with the heart of the patient, whereby, when the heart of the patient is in diastole, the pump forces blood from the first cannula into the patient, and when the heart is in systole, the pump removes blood from the patient via the first cannula.

4: An apparatus according to claim 1, wherein the pump controller includes at least a first input for receiving information relating to the actual heartbeat of the patient and a second input for receiving control signals from an operator interface, the pump controller being operable to utilize the information relating to the actual heartbeat of the patient and the control signals to control the pump in the second mode to synchronize the counter-pulsatile flow of the blood to the heart of the patient.

5: An apparatus according to claim 1, wherein the pump controller receives information relating to the actual heartbeat of the patient from an electrocardiogram.

6: An apparatus according to claim 1, wherein the pump controller receives information relating to the actual heartbeat of the patient from a pressure sensor sensing the blood pressure in the aorta or another artery.

7: An apparatus according to claim 1, wherein the pump controller further receives information relating to the operation of a pacemaker fitted to the patient.

8: An apparatus according to claim 1, wherein, when in the second mode, the pump is arranged to move between 0 and 200 ml of blood through the first cannula when counter-pulsating.

9: An apparatus according to claim 8, wherein, when in the second mode, the amount of blood to be moved through the first cannula can be controlled via the control signals from the operator interface.

10: An apparatus according to claim 1, wherein the pump is a roller-type pump.

11: The use of an apparatus according to claim 1, for providing short-term cardiac support after a heart has been restarted following surgery.

12: A method of using a cardiopulmonary bypass (CPB) pump (or heart-lung machine), the method comprising:
controlling a pump in a first mode to generate pulsatile flow of oxygenated blood through a cannula;
receiving signals from at least one of:
an electrocardiogram;
a pressure transducer; and
a pacemaker;
controlling the pump in a second mode to generate counter-pulsatile flow of blood through the cannula synchronized with the signals from the electrocardiogram, the pressure transducer and/or the pacemaker.

13: A method according to claim 12, wherein, when in the second mode, the pump is arranged to move between 0 and 200 ml (preferably 0-100 ml) of blood through the cannula when counter-pulsating.

14: A method according to claim 13, further comprising, when in the second mode, controlling the amount of blood to be moved through the cannula via control signals from an operator interface.
